(19) United States
(12) Patent Application Publication
(10) Pub. No.: US 2012/0077089 A1
(43) Pub. Date: Mar. 29, 2012

(54) POSITIVE ELECTRODE ACTIVE MATERIAL FOR LITHIUM PRIMARY CELL

(75) Inventors: Hideo SAKATA, Osaka (JP); Meiten KOH, Osaka (JP); Hitomi NAKAZAWA, Osaka (JP); Tomoyo SANAGI, Osaka (JP)

(73) Assignee: DAIKIN INDUSTRIES, LTD., Osaka (JP)

(21) Appl. No.: 13/245,938

(22) Filed: Sep. 27, 2011

(30) Foreign Application Priority Data
Sep. 28, 2010 (JP) 2010-217447

(51) Int. Cl. H01M 4/133; C07C 19/08

(52) U.S. Cl. 429/231.7; 570/134

(57) ABSTRACT

The present invention provides a positive electrode active material for a lithium primary cell. The positive electrode active material can reduce the internal resistance of the positive electrode of a lithium primary cell and can maintain the load characteristics and the discharge voltage not only at high temperatures but also at low temperatures. The positive electrode active material includes a fluoride of a low crystalline carbon.

### CROSS-REFERENCE TO RELATED APPLICATION

The present application claims priority under 35 U.S.C. §119 to Japanese Patent Application No. 2010-217447, filed Sep. 28, 2010. The contents of that application are incorporated herein by reference in their entirety.

### TECHNICAL FIELD

The present invention relates to a positive electrode active material for a lithium primary cell which gives a positive electrode having a low internal resistance; a positive electrode including the positive electrode active material; and a lithium primary cell having the positive electrode.

### BACKGROUND ART

Lithium primary cells are used widely as a power supply of devices such as mobile electronic equipment and tire air pressure sensors.

For positive electrode active materials for a lithium primary cell, materials mainly containing manganese dioxide or graphite fluoride are used. BR type cells containing graphite fluoride as a positive electrode active material have an advantage in that they have an internal resistance that is less likely to increase even at high temperatures.

However, BR type cells have a problem that they have low load characteristics and low discharge voltage at low temperatures (e.g. at −40° C.).

To solve the problem, the following methods, for example, have been proposed: a method of using a highly fluorinated graphite fluoride and a low-fluorinated graphite fluoride in combination (Patent Document 1); a method of fluorinating only the surface of graphite particles (Patent Document 2); a method of increasing the concentration ratio F/C of fluorine atoms to carbon atoms on the surface of the graphite fluoride (Patent Document 3); a method of using a graphite fluoride having a small particle size (Patent Document 4); a method of forming a carbon layer on the surface of the graphite fluoride particles (Patent Document 5); and a method of introducing a hydroxyl group or a carboxyl group to the surface of the graphite fluoride particles (Patent Document 6).

### CITATION LIST

### Patent Literature

Patent Document 1: JP 2006-236888 A
Patent Document 2: JP 2006-236891 A
Patent Document 3: JP 2009-152174 A
Patent Document 4: JP 2005-247679 A
Patent Document 5: JP 58-5966 A.
Patent Document 6: JP 2006-059732 A

### SUMMARY OF THE INVENTION

### Problems to be Solved by the Invention

The methods taught in those Patent Documents, however, have a problem that they require a process such as controlling the degree of fluorination, forming a carbon layer, or introducing a functional group, in addition to fluorinating graphite. Such a process leads to a high internal resistance of the positive electrode.

The present invention aims to provide a positive electrode active material for a lithium primary cell that can decrease the internal resistance of the positive electrode of a lithium primary cell, and maintain the load characteristics and the discharge voltage not only at high temperatures but also at low temperatures.

### Means for Solving the Problems

The present invention enables to achieve the above aim and relates to a positive electrode active material for a lithium primary cell, including a fluoride of a low crystalline carbon; a positive electrode including the positive electrode active material; and a lithium primary cell including the positive electrode, a negative electrode, and a nonaqueous electrolyte.

The present invention also relates to a fluoride of a low crystalline carbon, for a positive electrode active material for a lithium primary cell.

### Effect of the Invention

The present invention can provide a positive electrode active material for a lithium primary cell capable of decreasing the internal resistance of the positive electrode of a lithium primary cell, and maintaining the load characteristics and the discharge voltage not only at high temperatures but also at low temperatures.

### BRIEF DESCRIPTION OF DRAWINGS

FIG. 1 is a schematic cross-sectional view of a lithium primary cell according to one embodiment of the present invention.

### MODES FOR CARRYING OUT THE INVENTION

The positive electrode active material of the present invention includes a fluoride of low crystalline carbon.

The term “low crystalline carbon” herein refers to a carbon produced by heat-treating a carbon precursor at temperatures of 600° C. to 1500° C., preferably 1000° C. to 1400° C. A low crystalline carbon has a crystal structure having a high proportion of turbostratic structure and a very low proportion of graphite layer structure. The powder X-ray diffraction of such a carbon does not show the (101) diffraction peak, which means that the carbon atoms are less likely to be in the hexagonal graphite lattice. Examples of the low crystalline carbon include carbon blacks such as ketjen black, acetylene black, contact black, furnace black, and lamp black; nanocarbon materials such as carbon nanotubes and carbon fibers; activated carbon; and glassy carbon.

Among these, carbon blacks are preferable in terms of good electron conductivity, and ketjen black and acetylene black are more preferable.

Particularly preferable is ketjen black that is a carbon black in the form of hollow particles and is highly conductive. Such a carbon black has lower surface resistance than other low crystalline carbons in the case of having the same fluorine content, and thus can decrease the internal resistance of a positive electrode when used for the positive electrode.

Examples of commercially available products of ketjen black include Ketjenblack EC300J, Carbon ECP, Ketjenblack EC600JD, and Carbon ECP600JD produced by Lion Corporation.

A fluoride of a low crystalline carbon can be obtained by a method of bringing fluorine gas into direct contact with the low crystalline carbon, or a method of bringing hydrogen fluoride gas into direct contact with the low crystalline carbon. The temperature at which the low crystalline carbon is brought into contact with fluorine gas or hydrogen fluoride gas may be 0° C. to 500° C., and the duration of the contact may be 5 minutes to 48 hours.

The fluoride of a low crystalline carbon preferably has a fluorine content of 40.0% by mass or more in terms of large cell capacity, and 62.0% by mass or less in terms of good large current discharge. The maximum fluorine content is preferably 52.0% by mass, and more preferably 50.0% by mass in terms of large cell capacity. The minimum fluorine content is preferably 48.0% by mass, and more preferably 49.0% by mass in terms of good large current discharge.

Generally, carbon has electrical insulation properties upon fluorination. A fluoride of a high crystalline carbon material such as petroleum coke and graphite has higher electrical resistance than a fluoride of a low crystalline carbon material even if the fluorides have the same fluorine content, which is evident from the later-described results of examples and comparative examples.

The positive electrode active, material of the present invention which contains a fluoride of a low crystalline carbon may optionally further contain a fluoride of a conventionally known high crystalline carbon such as graphite to the extent that does not deteriorate the effects of the present invention.

The fluoride of a low crystalline carbon according to the present invention is preferably a high-temperature treated material. If a fluoride of a low crystalline carbon obtained by fluorinating a low crystalline carbon is subjected to a high-temperature treatment, free hydrofluoric acid adhering to the surface of the fluoride, that is, a cause of an increase in the resistance, can be removed, and the internal resistance of the cell can be decreased.

Examples of the high-temperature treatment include a method of heating a fluoride to a temperature of about 200° C. to 400° C. under stream of an inert gas such as nitrogen gas or in the air, for example. Here, the treatment time is suitably about 1 to 12 hours. The high-temperature treatment can greatly decrease the amount of free hydrofluoric acid of the fluoride.

The present invention also relates to a positive electrode for a lithium primary cell which includes the positive electrode active material of the present invention.

The positive electrode including the positive electrode active material of the present invention can be produced by a conventionally known method. That is, the positive electrode can be produced by, for example, mixing materials including the positive electrode active material of the present invention, a binder, and a conductive material, press-molding the mixture, and drying the molded product.

The binder may be a conventionally known material. Examples thereof include various polymers, including fluoropolymers such as polytetrafluoroethylene (PTFE), polyvinylidene fluoride (PVDF), modified PVDF, tetrafluoroethylene-hexafluoropropylene copolymer (FEP), tetrafluoroethylene-perfluoroalkyl vinyl ether copolymer (PFA), vinylidene fluoride-hexafluoropropylene copolymer, vinylidene fluoride-chlorotrifluoroethylene copolymer, ethylene-tetrafluoroethylene copolymer (ETFE), vinylidene fluoride-pentafluoropropylene copolymer, propylene-tetrafluoroethylene copolymer, ethylene-chlorotrifluoroethylene copolymer (ECTFE), and vinylidene fluoride-hexafluoropropylene-tetrafluoroethylene copolymer; styrene butadiene rubber (SBR), modified acrylonitrile rubber, ethylene-acrylic acid copolymers, and mixtures thereof. Among these, PTFE is preferable in terms of low resistance and good moldability. The amount of the binder in the positive electrode is preferably 1% by mass or more and 10% by mass or less.

The conductive material may be a conventionally known material, and examples thereof include non-fluorinated carbon blacks such as non-fluorinated ketjen black, non-fluorinated acetylene black, non-fluorinated contact black, non-fluorinated furnace black, and non-fluorinated lamp black. Among these, non-fluorinated ketjen black is preferable in terms of good electrical conductivity. The amount of the conductive material in the positive electrode is preferably 1% by mass or more and 10% by mass or less.

The amount of the fluoride of a low crystalline carbon in the positive electrode of the present invention is preferably 80% by mass or more, and more preferably 90% by mass or more in terms of large cell capacity. The amount is preferably 95% by mass or less, and more preferably 93% by mass or less in terms of not too high an internal resistance.

The present invention also relates to a lithium primary cell including the positive electrode of the present invention, a negative electrode, and a nonaqueous electrolyte.

The negative electrode may be a material commonly used as the negative electrode of lithium primary cells, such as metallic lithium and lithium alloys. Examples of the lithium alloys include Li—Al alloy.

The nonaqueous electrolyte may be a material commonly used as the nonaqueous electrolyte of lithium primary cells which is produced by dissolving an electrolyte salt in an organic solvent. Examples of the organic solvent include propylene carbonate, dimethyl carbonate, diethyl carbonate, 2-methyltetrahydrofuran, dioxolane, tetrahydrofuran, methyl ethyl carbonate, dipropyl carbonate, ethylene carbonate, γ-butyrolactone, dimethyl sulfoxide, acetonitrile, formamide, dimethylformamide, triglyme (tri(ethylene glycol) dimethyl ether), diglyme (diethylene glycol dimethyl ether), DME (glyme, 1,2-dimethoxyethane, or ethylene glycol dimethyl ether), nitromethane, and mixtures thereof. Examples of the electrolyte salt include lithium hexafluorophosphate, lithium hexafluoroarsenate, lithium bis(trifluoromethylsulfonyl)imide, lithium trifluoromethanesulfonate, lithium tris(tetrafluoromethylsulfonyl)methide, lithium tetrafluoroborate, lithium perchlorate, and lithium tetrachloroaluminate.

Lithium primary cells generally have a separator between the positive electrode and the negative electrode. Examples of the separator include, but not particularly limited to, microporous polyethylene films, microporous polypropylene films, microporous ethylene-propylene copolymer films, microporous polypropylene/polyethylene two-layer films, microporous polypropylene/polyethylene/polypropylene three-layer films.

The above respective components are assembled into the lithium primary cell of the present invention by a commonly used method.

### EXAMPLES

Hereinafter, the present invention will be described in more detail based on the examples which, however, are not intended to limit the scope of the present invention.

The fluorine content was measured by the following method.

### (Measurement of Fluorine Content)

A powdery sample of a fluorinated low crystalline carbon is decomposed by heating at 1200° C. with an automatic burner (AQF-100 produced by Mitsubishi Chemical Corporation) such that the generated gas is absorbed by a predetermined amount of a hydrogen peroxide solution. The ion concentration of the fluoride in the obtained absorbing solution (measurement sample) is quantified by ion chromatography with ICS-1500 produced by Nippon Dionex K.K. The mass ratio of the fluorine atoms and the carbon atoms in the fluorinated low crystalline carbon is determined based on the fluorine content in the measurement sample (absorbing solution) and the amount of the fluorinated low crystalline carbon, whereby the fluorine content (% by mass) in the fluorinated low crystalline carbon is calculated.

Preparation 1 (Production of Fluorinated Ketjen Black Having Fluorine Content of 61.0% by Mass)

A constant-temperature bath capable of circulating fluorine gas was charged with 1 kg of ketjen black (Ketjenblack EC600J produced by Lion Corporation), and the ketjen black was allowed to react with fluorine gas for 12 hours under the conditions of a fluorine gas pressure of 0.5 atm (5.07×10⁴ Pa) and a heating temperature of 400° C. Thereby, fluorinated ketjen black having a fluorine content of 61.0% by mass was produced.

An amount of 1 g of the obtained fluorinated ketjen black was mixed into 20 g of γ-butyrolactone, and the mixture was left to stand at 100° C. for one week. Thereafter, the fluorinated ketjen black was removed by filtration, and the concentration of the free hydrofluoric acid in the filtrate was measured by an F ion meter (ORION EA940 produced by Thermo Fisher Scientific). The measured concentration was 0.034% by mass.

Preparation 2 (Production of Fluorinated Ketjen Black Having Fluorine Content of 48.0% by Mass)

A fluorinated ketjen black having a fluorine content of 48.0% by mass was produced by the same procedure as that in Preparation 1, except that the reaction time was changed to two hours. The concentration of the free hydrofluoric acid of the obtained fluorinated ketjen black was measured as in Preparation 1. The measured concentration was 0.023% by mass.

Preparation 3 (Production of Fluorinated Ketjen Black Having Fluorine Content of 54.8% by Mass)

A fluorinated ketjen black having a fluorine content of 54.8% by mass was produced by the same procedure as that in Preparation 1, except that the reaction time was changed to six hours. The concentration of the free hydrofluoric acid of the obtained fluorinated ketjen black was measured as in Preparation 1. The measured concentration was 0.030% by mass.

Preparation 4 (Production of High-Temperature Treated Material)

The fluorinated ketjen black having a fluorine content of 61.0% by mass produced in Preparation 1 was left to stand at 400° C. for 12 more hours under nitrogen stream for the high-temperature treatment, so that a high-temperature treated fluorinated ketjen black was obtained. The concentration of the free hydrofluoric acid of the obtained high-temperature treated fluorinated ketjen black was measured as in Preparation 1. The measured concentration of the free hydrofluoric acid was 0.008% by mass, greatly decreased from the concentration measured before the high-temperature treatment.

Preparation 5 (Production of High-Temperature Treated Material)

The fluorinated ketjen black having a fluorine content of 61.0% by mass produced in Preparation 1 was left to stand at 400° C. for one more hour under nitrogen stream for the high-temperature treatment, so that a high-temperature treated fluorinated ketjen black was obtained. The concentration of the free hydrofluoric acid of the obtained high-temperature treated fluorinated ketjen black was measured as in Preparation 1. The measured concentration of the free hydrofluoric acid was 0.019% by mass, greatly decreased from the concentration measured before the high-temperature treatment.

Preparation 6 (Production of High-Temperature Treated Material)

The fluorinated ketjen black having a fluorine content of 61.0% by mass produced in Preparation 1 was left to stand at 200° C. for one more hour under nitrogen stream for the high-temperature treatment, so that a high-temperature treated fluorinated ketjen black was obtained. The concentration of the free hydrofluoric acid of the obtained high-temperature treated fluorinated ketjen black was measured as in Preparation 1. The measured concentration of the free hydrofluoric acid was 0.025% by mass, greatly decreased from the concentration measured before the high-temperature treatment.

Comparative Preparation 1 (Production of Fluorinated Petroleum Coke Having Fluorine Content of 61.0% by Mass)

A constant-temperature bath capable of circulating fluorine gas was charged with 1 kg of petroleum coke (GL Coke produced by Great Lakes Company) which is a high crystalline carbon, and the petroleum coke was allowed to react with fluorine gas for eight hours under the conditions of a fluorine gas pressure of 0.5 atm (5.07×10⁴ Pa) and a heating temperature of 400° C. Thereby, fluorinated petroleum coke having a fluorine content of 61.0% by mass was produced.

The concentration of the free hydrofluoric acid of the obtained fluorinated petroleum coke was measured as in Preparation 1. The measured concentration was 0.026% by mass.

Comparative Preparation 2 (Production of Graphite Fluoride Having Fluorine Content of 54.8% by Mass)

A constant-temperature bath capable of circulating fluorine gas was charged with 1 kg of artificial graphite (HAG-15 produced by Lonza Group AG) which is a high crystalline carbon, and the artificial graphite was allowed to react with fluorine gas for 54 hours under the conditions of a fluorine gas pressure of 0.5 atm (5.07×10⁴ Pa) and a heating temperature of 400° C. Thereby, graphite fluoride having a fluorine content of 54.8% by mass was produced.

The concentration of the free hydrofluoric acid of the obtained graphite fluoride was measured as in Preparation 1. The measured concentration was 0.014% by mass.

### Examples 1 to 6 and Comparative Examples 1 and 2

### Production of Lithium Primary Coin Cell

### (Production of Positive Electrode)

To 90 parts by mass of the fluoride produced as a positive active material in each of Preparations 1 to 6 and Comparative Preparations 1 and 2 were added 5 parts by mass of ketjen black as a conductive material, 5 parts by mass (solids content) of a PTFE dispersion (D-210C produced by Daikin Industries, Ltd.) as a binder, pure water, and a small amount of ethanol. The materials were mixed, and the mixture was dried and then crushed into a powdery product. The powdery product was pressure-molded into a disk pellet having a diameter of 16 mm and a thickness of 3 mm. The pellet was dried at a high temperature (at 200° for four hours) to remove water in the pellet. Thereby, a positive electrode was produced.

### (Production of Negative Electrode)

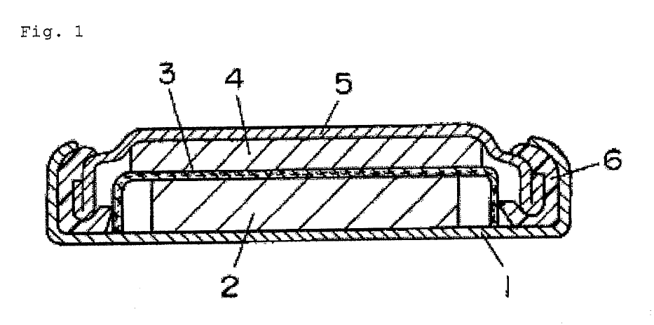

A negative electrode was produced by punching a 1.0-mm thick lithium foil into a disk having a diameter of 18 mm, and then press-bonding the resulting lithium foil to the inner surface of a sealing plate (the symbol 5 in FIG. 1) such that the centers of the lithium foil and the plate were the same.

### (Preparation of Nonaqueous Electrolyte)

The electrolyte used was prepared by dissolving 1 mole of LiBF₄ as the electrolyte salt in γ-butyrolactone as the solvent.

### (Assembly of a Lithium Primary Coin Cell)

A lithium primary coin cell (diameter: 23 mm, thickness: 2 mm) containing a nonaqueous electrolyte as illustrated in FIG. 1 was produced. In FIG. 1, a cell case 1 functions also as a positive electrode terminal and is made of stainless steel. A positive electrode 2 is a pellet obtained by pressure-molding a powder mixture of one of the fluorides produced in the preparations and comparative preparations, a conductive material, and a binder. A separator 3 is a nonwoven fabric made of polyethylene. A negative electrode 4 is metal lithium. A sealing plate 5 is a disk plate made of stainless steel and functions also as a negative electrode terminal. The cell is sealed with gaskets 6.

The internal resistance (Ω) at 1 kHz and low-temperature discharging characteristics of the respective produced lithium primary coin cells were determined as follows. Table 1 shows the results.

### (Measurement of Internal Resistance)

The lithium primary coin cell was left to stand at −40° C., and the internal resistance at 1 kHz was measured by an LCR meter 4263B produced by Agilent Technologies International Japan, Ltd.

### (Measurement of Low-Temperature Discharging Characteristics)

The lithium primary coin cell was allowed to stand at −25° C., and the cell was discharged in a repeated pattern of 10-mA discharge for 100 ms per minute. After 300 hours, the voltage (V) right before the pulse current flow and the pulse discharge voltage were measured. The measurement was performed for ten samples of the cell and the average calculated therefrom was used for evaluation.

TABLE 1

| | | Pulse | Voltage (V) |
|---|---|---|---|
| | Internal | discharge | right before pulse |
| | resistance (Ω) | voltage (V) | current flow |
| Example 1 | 105 | 1.80 | 2.70 |
| Example 2 | 93 | 1.93 | 2.85 |
| Example 3 | 97 | 1.85 | 2.80 |
| Example 4 | 98 | 1.87 | 2.78 |
| Example 5 | 100 | 1.84 | 2.75 |
| Example 6 | 101 | 1.82 | 2.74 |
| Comparative | 116 | 1.70 | 2.65 |
| Example 1 | | | |
| Comparative | 130 | 1.62 | 2.60 |
| Example 2 | | | |

The results in Table 1 show that the cell including the fluorinated ketjen black which is a fluoride of a low crystalline carbon had a lower internal resistance than the cells including the fluorinated petroleum coke or graphite fluoride which are fluorides of high crystalline carbons. Further, the results also show that the cells subjected to the high-temperature treatment in Examples 4 to 6 had higher pulse voltages and better low-temperature load characteristics than the cell produced in Example 1.

### EXPLANATION OF NUMERALS

1 Positive electrode case (cell case)
2 Positive electrode pellet (positive electrode)
3 Separator
4 Lithium (negative electrode)
5 Sealing plate
6 Gasket

1. A positive electrode active material for a lithium primary cell, comprising
a fluoride of a low crystalline carbon.

2. The positive electrode active material according to claim 1,
wherein the fluoride of a low crystalline carbon is a high-temperature treated material.

3. The positive electrode active material according to claim 1,
wherein the fluoride of a low crystalline carbon has a fluorine content of 40.0% by mass to 62.0% by mass.

4. The positive electrode active material according to claim 1,
wherein the low crystalline carbon is carbon black.

5. The positive electrode active material according to claim 1,
wherein the low crystalline carbon is ketjen black.

6. A positive electrode for a lithium primary cell, comprising
the positive electrode active material according to claim 1.

7. A lithium primary cell, comprising
the positive electrode according to claim 6,
a negative electrode, and
a nonaqueous electrolyte.

8. A fluoride of a low crystalline carbon, for a positive electrode active material for a lithium primary cell.
